(19) United States
(12) Patent Application Publication
(10) Pub. No.: US 2007/0082922 A1
(43) Pub. Date: Apr. 12, 2007

(54) Huperzine a prodrugs and uses thereof

(76) Inventor: Bai-Chuan Pan, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/244,932

(22) Filed: Oct. 6, 2005

(51) Int. Cl. A61K 31/473; C07D 221/22

(52) U.S. Cl. 514/290; 546/97

(57) ABSTRACT

Disclosed are huperzine A prodrugs and method of synthesis thereof. The invention further relates to methods of treating, preventing or reversing neurodegenerative diseases, such as, Alzheimer's Disease and neuronal dysfunctions, such as, memory impairment using a pharmaceutical composition comprising a huperzine A prodrug as disclosed herein.

### BACKGROUND OF THE INVENTION

Alzheimer's Disease (AD) is a chronic, neurodegenerative disorder characterized by a loss of cognitive ability and severe behavioral abnormalities in a patient, leading to the eventual death of the patient. There are currently 2.5 to 4.0 million AD patients in the U.S. and 17 to 25 million worldwide. It is the fourth leading cause of death in Western cultures, preceded only by heart disease, cancer, and stroke.

One of the problems of Alzheimer's disease is cholinergic dysfunction resulting from a deficiency in the neurotransmitter acetylcholine. Acetylcholine plays a fundamental role in memory. Alzheimer's disease results in marked damage to the cholinergic neurons in the brain as well as damage to the enzymes: acetylcholinesterase and choline acetyltransferase [Bartus, R. T.;Dean, R. L.; Beer, B.; Lippa, A. S. Science, 1982, 217, 408.] Alzheimer's disease can be treated by inhibition of acetylcholinesterase which results in an increase in acetylcholine levels in the brain. ARICEPT® is an acetylcholinesterase inhibitor which has been approved by the FDA for decelerating the rate of decline in Alzheimer's disease patients. However, ARICEPT® is only effective for a limited period of time and it does not function over the entire patient population. Currently, there is no definitive treatment, or cure for this devastating disease.

Huperzine A is an alkaloid isolated from Chinese herb Lycopodium Serratum Thumb. [Liu, J. S., Zhu, Y. L.; Yu, C. M.; Zhou, Y. Z.: Han, Y. Y.: Wu, F. W.: QI, B. F. Can. J. Chem. 1986,64, 837]. The chemical structure of huperzine A, also known as 5-amino-11-ethylidene-5,6,9,10-tetrahydro-7-methyl-5,9-methylenecyclocta[b]pyridyl-2-(1H)-one, is as shown below as structural formula (I).

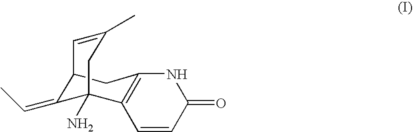

Huperzine A is a potent reversible inhibitor of acetylcholinesterase with high specificity and a very low rate of dissociation from the enzyme [Ashani, Y.; Peggins, J. O. III: Doctor, B. P. Biochem. Biophys. Res. Commun. 1992, 184, 719-726.] Huperzine A may also increase the level of neurotransmitter acetylcholine in the central nervous system. It is currently under clinical trial for the treatment of Alzheimer's disease [Kozikowski, A. P.: J. Heterocyclic Chem. 1990, 27, 97. Bai, D. Pure and Appl. Chem. 1993, 65, 1103.].

Recently a series of amide and Schiff base derivatives of huperzine A have been synthesized [U.S. Pat. No. 5,929,084 and U.S. Pat. No. RE38,460 E]. The amide derivatives of huperzine A are inactive. The Schiff bases of huperzine A are active due to the activation by nonenzymatic hydrolysis. However, the Schiff base derivatives usually are inherently unstable, and may cause stability problems and therefore would be unsuitable for use as a drug.

Therefore, there is a need for new compounds and pharmaceutical compositions comprising such compounds to overcome the disadvantages of the currently available compounds in the treatment of Alzheimer's disease.

### SUMMARY OF THE INVENTION

The present invention relates to huperzine A prodrugs and their use to inhibit cholinesterase activity and to treat, prevent or reverse neurodegenerative diseases, such as, Alzheimer's Disease and other neuronal dysfunctions, such as memory impairment. In particular, the compounds disclosed herein participate in in vivo reactions resulting in conversion to active huperzine A. Huperzine A has been shown to improve cognitive function in mammals [Jiang H. et al., Current Medicinal Chemistry, 10: 1241-1253 (2003)], the teachings of which are incorporate herein by reference. A specific advantage of the prodrug compounds described herein is their ability to cross the blood-brain barrier and effectively inhibit cholinesterase (e.g., acetylcholinesterase) activity.

In one embodiment, the present invention relates to compounds represented by the formula (II): 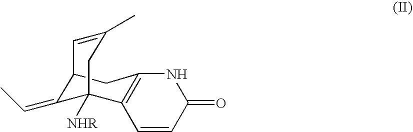
or pharmaceutically salts and solvates (e.g., hydrates) thereof.

R is
i) —C(O)C(NR₃R₄)R₁R₂ or —C(O)C[NR₃C(O)C(NR₃R₆)R₁R₂]R₁R₂; 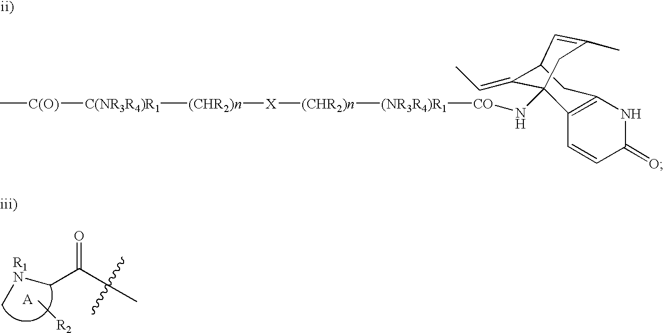
A is a 5, 6 or 7 membered nitrogen containing heterocyclic group;
iv) —CR₃R₄OC(O)R₅;
v) —C(O)OR₅; or

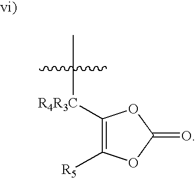

Each R₁ is independently H, C1-20-alkyl, C1-20-aralkyl, aryl, C2-20-alkenyl, C2-20-aralkenyl, C2-20-alknyl, C2-20-aralknyl, carbocyclylalkyl, heterocycloalkyl, C3-20-cycloalkyl, C1-20-hydroxyaralkyl, C1-20-hydroxyalkyl, C1-20-nitroaralkyl, C1-20-nitroalkyl, C1-20-alkoxyalkyl, C1-20-thioalkyl, C1-20-alkylthioalkyl, C1-20-aminoalkyl, C1-20-carboxyalkyl, C1-20-alkoxycarboalkyl, C1-20-aminocarboalkyl, C1-20-guanidinoalkyl, heterocyclyl, C1-20-heterocycloalkyl, C1-20-cycloaminoalkyl.

Each R₂, R₃ and R₄ are independently —H, C1-4-alkyl, C1-4-aralkyl, aryl, C2-4-alkenyl, C2-4-alknyl, or —NR₃R₄ forms a 4-6 membered nitrogen containing heterocyclic ring, or —CR₃—R₄ forms a 4-6 membered carbocyclic ring.

Each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino.

Each R₆ is independently R₄ or —C(O)C(NR₃R₄)R₁R₂.

Each n is independently 1, 2, 3, 4, 5, 6, 7 or 8.

X is —O—, —N(R₂)—, —S—, —(CR₂R₃)ₙ, —O—(CR₂R₃)ₙ—O—, —S—(CR₂R₃)ₙ—S—, —N(R₂)—(CR₂R₃)ₙ—N(R₂)—, —O—C(O)—(CR₂R₃)ₙ—C(O)—O— or —C(O)—O—(CR₂R₃)ₙ—O—C(O)—.

In another embodiment, the present invention relates to processes for preparation of compounds of represented by formula (II).

In yet another embodiment, the present invention relates to pharmaceutical compositions comprising compounds represented by formula (II) and pharmaceutically acceptable carriers or excipients.

In yet another embodiment, the present invention relates to methods of treating, preventing or reversing a neurodegenerative disease, such as, Alzheimer's Disease, or a neuronal dysftunction, such as, memory impairment comprising administering to a subject, in need thereof, a therapeutically effective amount of a compound represented by formula (II) or a pharmaceutically acceptable salt or solvate (e.g., hydrate) thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present invention provides compounds encompassed by formula (II), or pharmaceutically acceptable salts and solvates thereof:

R is
i) —C(O)C(NR₃R₄)R₁R₂ or —C(O)C[NR₃C(O)C(NR₃R₆)R₁R₂]R₁R₂; 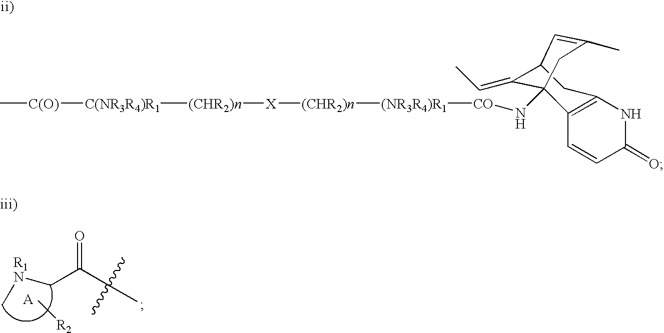
A is a 5, 6 or 7 membered nitrogen containing heterocyclic group
iv) —CR₃R₄OC(O)R₅;
v) —COOR₅; or

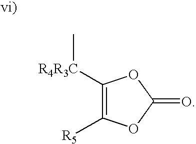

In certain embodiments, R is —C(O)C(NR₃R₄)R₁R₂ or —C(O)C[NR₃C(O)C(NR₃R₆)R₁R₂]R₁R₂. In certain particular embodiments R is a synthetic or naturally occurring α-amino-acyl group. In certain particular embodiments, R is a synthetic or naturally occurring peptidyl, dipeptidyl or tripeptidyl group.

In certain other embodiments, R is:

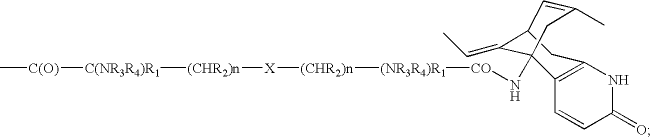

In certain other embodiments, R is:

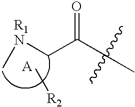

wherein A is a 5, 6 or 7 membered nitrogen containing heterocyclic group. In certain other particular embodiments R is a natural α-amino-acyl group. In certain embodiments A is L-pyrolyl.

In certain other embodiments, R is —CR₃R₄OC(O)R₅.

In certain other embodiments, R is —COOR₅.

In certain other embodiments R is

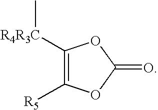

Each R₁ is independently H, C1-20-alkyl, C1-20-aralkyl, aryl, C2-20-alkenyl, C2-20-aralkenyl, C2-20-alknyl, C2-20-aralknyl, carbocyclylalkyl, heterocycloalkyl, C3-20-cycloalkyl, C1-20-hydroxyaralkyl, C1-20-hydroxyalkyl, C1-20-nitroaralkyl, C1-20-nitroalkyl, C1-20-alkoxyalkyl, C1-20-thioalkyl, C1-20-alkylthioalkyl, C1-20-aminoalkyl, C1-20-carboxyalkyl, C1-20-alkoxycarboalkyl, C1-20-aminocarboalkyl, C1-20-guanidinoalkyl, heterocyclyl, C1-20-heterocycloalkyl, C1-20-cycloaminoalkyl.

Each R₂, R₃ and R₄ are independently —H, C1-4-alkyl, C1-4-aralkyl, aryl, C2-4-alkenyl, C2-4-alknyl, or —NR₃R₄ forms a 4-6 membered nitrogen containing heterocyclic ring, or —CR₃—R₄ forms a 4-6 membered carbocyclic ring. In certain embodiments R₂, R₃ and R₄ are —H.

Each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino.

Each R₆ is independently R₄ or —C(O)C(N₃R₄)R₁R₂.

In certain embodiments each R₆ is independently —C(O)C[NR₃C(O)C(NR₃R₄)R₁R₂]R₁R₂ or —C(O)C{NR₃C(O)C[NR₃C(O)C(NR₃R₄)R₁R₂]R₁R₂}R₁R₂.

Each n is independently 1, 2, 3, 4, 5, 6, 7 or 8.

X is —O—, —N(R₂)—, —S—, —(CR₂R₃)ₙ, —O—(CR₂R₃)ₙ, —O—, —S—(CR₂R₃)ₙ—S—, —N(R₂)—(CR₂R₃)ₙ—N(R₂)—, —O—C(O)—(CR₂R₃)ₙ—C(O)—O— or —C(O)—O—(CR₂R₃)ₙ—O—C(O)—.

In certain other particular embodiments the compound of the present invention is a prodrug of huperzine A which is the product of, for example, a condensation reaction of synthetic and/or natural amino acids (including all stereoisomers) with (−)-huperzine A. In certain other particular embodiments the compound of the present invention is a prodrug of huperzine A which is the product of, for example, a condensation reaction of peptides (dipeptides, tripeptides, and the like) comprised of synthetic and/or natural amino acids (including all stereoisomers) with (−)-huperzine A.

The term “C1-20-alkyl” refers to substituted or unsubstituted saturated hydrocarbon groups, including straight-chain alkyl and branched-chain alkyl that contain from 1-20 carbons in the chain, including haloalkyl groups such as trifluoromethyl and 2,2,2-trifluoroethyl, and so on.

The term “C1-20-aralkyl” refers to a C1-20alkyl group substituted with an aromatic group.

The term “amino” refers to both substituted and unsubstituted amines and salts thereof.

The term “C1-20-aminoalkyl” refers to a C1-20-alkyl group substituted with an amino group.

The term “C1-20-gunidinoalkyl” refers to a C1-20-alkyl group substituted with a guanidino group.

The term “C1-20-hydroxyaralkyl” refers to a C1-20-alkyl group substituted with an aromatic group and a hydroxy group.

The term “C1-20-nitroaralkyl” refers to a C1-20-alkyl group substituted with an aromatic group and a nitro group.

The term “C1-20-nitroalkyl” refers to a C1-20-alkyl group substituted with a nitro group.

The term “alkoxy” refers to an alkyl group having an oxygen attached thereto. Representative alkoxy groups include methoxy, ethoxy, ter-butoxy and the like.

The term “C1-20-alkoxyalkyl” refers to a C-1-20-alkyl group substituted with an alkoxy group thereby forming an ether.

The term “C1-20-thioalkyl” refers to a C1-20-alkyl group substituted with a thiol group.

The term “alkylthio” refers to an alkyl group having a sulfur attached thereto.

The term “C1-20-alkylthioalkyl” refers to a C1-20-alkyl group substituted with an alkylthio group thereby forming a sulfide.

The term “C1-20-alkoxycarbonylalkyl” refers to a C1-20-alkyl group substituted with a carboxylic esters group.

The term “C1-20-aminocarbonylalkyl” refers to a C1-20-alkyl group substituted with an amide group.

The term “C2-20-alkenyl” and “C2-20-alkynyl” refer to substituted or unsubstituted aliphatic groups analogous in length and possible substitution of the alkyl described above, but those contain at least one double or triple bond respectively.

The term “C2-20-aralkenyl” and “C2-20-aralkynyl” refer to C2-20-alkenyl and C2-20-alkynyl group substituted with an aryl group.

The term “aryl” as used herein includes 5, 6 or 7 membered substituted or unsubstituted single-ring aromatic group in which each atom of the ring is carbon.

The term “aryl” also includes polycyclic ring systems having two or more cyclic rings in which two or more carbons are common to two adjoining rings wherein at least one of the rings is aromatic e.g., the other cyclic ring can be cycloalkyl, cycloalkenyl, cycloalkynyl, heteroaryls and/or heterocyclyl. Aryl groups can be substituted or unsubstituted, examples include benzene, naphthalene, phenanthrene, aniline, phenol, and the like.

The term “carbocyclyl” refers to a nonaromatic substituted or unsubstituted ring in which each atom of the ring is carbon. It also includes polycyclic ring systems having two or more cyclic rings in which two or more carbons are common to two adjoining rings wherein at least one of the rings is carbocyclic e.g. the other cyclic ring can be cycloalkyl, cycloalkenyl, cycloalkynyl, aryl, heteroaryl, and/or heterocyclyl.

The term “C1-20-carbocyclylalkyl” refers to a C1-20-alkyl group substituted with a carbocyclyl group.

The term “heterocyclyl” refers to nonaromatic substituted or unsubstituted 3-10 membered ring structure, more preferably 3-7 membered structure comprising one to four hetero atoms. It also includes polycyclic ring systems having two or more cyclic rings in which two or more atoms are common to two adjoining rings wherein at least one of the rings is heterocyclic e.g. the other cyclic ring can be cycloalkyl, cycloalkenyl, cycloalkynyl, aryl, heteroaryl and/or heterocyclyl. Heterocyclyl groups include for example indol, imidazole, piperidine, piperizine, pyrrolidine, morpholine, lactone, lactames and the like.

The term “C1-20-heterocyclylalkyl” refers to a C1-20-alkyl group substituted with a heterocyclyl group.

In certain embodiments, the starting material (not huperzine A) for the synthesis of the prodrug of huperzine A contains one or more than one chiral centers. All stereoisomers of the prodrug of huperzine A are encompassed herein. However, at times it may be desirable to select optically active compounds for certain uses. Optically active compounds can be isolated by techniques well known in the art.

The compounds of the present invention can be used to inhibit cholinesterase activity (e.g., acetylcholinesterase activity). The inhibition of said cholinesterase activity can be accomplished within an individual or within a cell, such as cells in culture. For example, cholinesterase activity can be inhibited in a subject in need thereof by administering to the subject a therapeutically effective amount of a compound disclosed herein, in particular a compound represented by Structural Formula (II) or a pharmaceutically acceptable salt or solvate thereof.

As used herein the term “subject” encompasses mammals, specifically humans.

In one embodiment the present invention pertains to treating, preventing or reversing a neurodegenerative disease, such as, Alzheimer's Disease in a subject in need thereof, or for treating or preventing or reversing a neuronal dysfunction in a subject in need thereof comprising administering to said subject a therapeutically effective amount of a compound represented by formula (II) or a pharmaceutically acceptable salt or solvate thereof.

Examples of neuronal dysfunction treated, prevented, or reversed using the compounds and compositions of the present invention include, but are not limited to, memory impairment; cognitive decline characterized by concentration loss; memory-acquisition loss; information-storage or retrieval loss; neuronal disorders such as cognitive decline associated with aging and minimal cognitive impairment; and dysfunction resulting from severe neurodegenerative disorders such as Alzheimer's disease.

The methods of the present invention can provide a prophylactic or therapeutic neuroprotective effect. For example, in another embodiment, the invention comprises a method of providing neuroprotection in a subject, said method comprising administering to a subject in need thereof a compound of compositions described herein, in particular a compound represented by formula (II) or a pharmaceutically acceptable salt or solvate thereof, in an amount effective to provide neuroprotection. As a result of such neuroprotection, the effects of neuronal dysfunction on neurodegenerative diseases can be alleviated, either partially or completely. Such alleviation in a subject can be determined by tests well known to those of skill in the art.

The compounds of the present invention can be tested for biological activity (e.g., activity in inhibiting cholinesterase or treating, preventing or reversing neurodegenerative disorders as described herein) using techniques known to those of skill in the art. For example, the compounds of the present invention can be tested in vivo using appropriate animal models, such as, mice, as described in U.S. Pat. No. 5,720,936. Other assays suitable for determining the activity of the claimed compounds are described in U.S. Pat. Nos. 4,929731 and RE 38,460, the teachings each of which are herein incorporated by reference. The compounds of the present invention are converted or metabolized (e.g., hydrolyzed) to huperzine-A, and accordingly if conversion to huperzine-A is demonstrated in vitro under appropriate physiological conditions, it is reasonable to assume that the prodrug will be converted to huperzine-A under similar physiological conditions in vivo. Thus, biological activity of the compounds of the present invention, and their efficacy for methods of treatment, can be evaluated.

The compositions of the present invention can be formulated as any one or more of the active compounds described herein and a physiologically acceptable carrier (also referred to as a pharmaceutically acceptable carrier or solution or diluent). Such carriers and solutions include pharmaceutically acceptable salts and solvates of compounds of the instant invention, and mixtures comprising two or more of such compounds, pharmaceutically acceptable salts of the compounds and pharmaceutically acceptable solvates of the compounds.

Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

The term “pharmaceutically acceptable salt” refers to salt forms that are pharmacologically suitable for or compatible with the treatment of patients, in particular, humans. If the inventive compound is a base, the desired pharmaceutically acceptable salt may be prepared by a suitable method available in the art, for example, treatment of the free base with an inorganic acid, such as hydrochloric acid, hydrobromic acid, sulfuric acid, nitric acid, phosphoric acid (e.g. the hydrochloride, hydrobromide, sulfate, nitrate, phosphate, salts etc.); and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, (e.g. the acetate, maleate, formate, trifluoroacetate, tartrate, methanesulfonate, benzenesulfonate, p-toluenesulfonate, salts etc.) or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of the suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The term “solvate” as used herein means a compound of the invention, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The compounds of formula (II) can be prepared in the form of their hydrates, such as hemihydrate, monohydrate, dihydrate, trihydrate, tetrahydrate and the likes or as solvates.

The compounds of formula (II), or pharmaceutically acceptable salts and solvates thereof, of the invention are suitably formulated into pharmaceutical or radiopharmaceutical compositions for administration to human subjects in a biologically compatible form suitable for administration in vivo. Accordingly, in another aspect, the present invention provides a pharmaceutical composition comprising a compound of formula (II), or pharmaceutically acceptable salts and solvates thereof, of the invention in admixture with a suitable diluent or carrier.

The term “effective amount” or “therapeutically effective amount” as used herein is that amount sufficient to effect desired results, including clinical results, and, as such, an “effective amount” depends upon the context in which it is being applied. For example, determination of an effective amount of a claimed compound useful for the treatment of memory impairment disorders in a subject can be based on tests such as those described in U.S. Pat. No. RE 38,460, the teachings of which are incorporated herein by reference.

The term “subject” as used herein includes all members of the animal kingdom including human. The subject is preferably a human.

The compounds for use in the method of the invention can be formulated for administration by any suitable route, such as oral or parenteral, for example, transdermal, transmucosal (e.g., sublingual, lingual, (trans)buccal), vaginal (e.g., trans- and perivaginally), (intra)nasal and (trans)rectal), subcutaneous, intramuscular, intradermal, intra-arterial, intravenous, inhalation, and topical administration.

Suitable compositions and dosage forms include tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays, dry powders or aerosolized formulations. It is preferred that the compounds are orally administered. Suitable oral dosage forms include, for example, tablets, capsules or caplets prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated, e.g., to provide for ease of swallowing or to provide a delayed release of active, using suitable methods. Liquid preparation for oral administration can be in the form of solutions, syrups or suspensions. Liquid preparations (e.g., solutions, suspensions and syrups) are also suitable for oral administration and can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates).

The dosage regimen utilizing the compounds of formula (II) can be selected in accordance with a variety of factors including age, weight and sex of the subject being treated; the disease being treated; the severity of the condition to be treated; the route of administration; the renal and hepatic function of the subject; and the particular compound or salt thereof employed. The skilled artisan can readily determine and prescribe the effective amount of the compound of formula (II) required to treat, for example, to prevent, inhibit (fully or partially) or arrest the progress of the disease.

It is understood that the total dosage amount per day can be administered in a single dose or can be administered in multiple dosings such as twice, three or four times per day. The compounds for use in the methods of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The compounds of the present invention can also be administered in conjunction with other currently used drugs to augment or supplement their therapeutic effects.

In another embodiment the present invention relates to kits for the treatment, prevention or reversal of neuronal dysfunction or a neurodegenerative disease wherein the kit, comprises an effective amount of one or more compounds of formula II, or pharmaceutically acceptable salts and solvates thereof.

Such kits are designed to give sterile products suitable for human administration, e.g. direct injection into the bloodstream. Suitable kits comprise containers (e.g. septum-sealed vials) containing the compound of formula (II).

The kits may optionally further comprise additional components such as radioprotectants, antimicrobial preservatives, pH-adjusting agents or fillers.

The term “radioprotectant” is defined herein as a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof.

The term “antimicrobial preservative” is defined herein as an agent which inhibits the growth of potentially harmful micro-organisms such as bacteria, yeasts or molds. The antimicrobial preservative may also exhibit some bactericidal properties, depending on the dose. The role of the antimicrobial preservative(s) encompassed by the present invention is to inhibit the growth of any such micro-organism in the pharmaceutical composition post-reconstitution. The antimicrobial preservative may, however, also optionally be used to inhibit the growth of potentially harmful micro-organisms in one or more components of the kit of the present invention prior to reconstitution. Suitable antimicrobial preservatives include: the parabens, i.e., ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit components is within acceptable limits (approximately pH 4.0 to 10.5) for human administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the compounds of the present invention are employed in acid salt form, the pH-adjusting agent may optionally be provided in a separate vial or container, so that the user of the kit can adjust the pH as part of a multi-step procedure.

The term “filler” is defined herein as a pharmaceutically acceptable bulking agent that may facilitate material handling during production and lyophilisation. Suitable fillers include inorganic salts such as sodium chloride, and water soluble sugars or sugar alcohols such as sucrose, maltose, mannitol or trehalose. The present invention also pertains to methods for preparing compounds represented by formula (II). In one embodiment of the present invention, compounds of formula (II), when R is —C(O)C(NH₂)R₁R₂ can be synthesized by any method known in the art for the synthesis of peptides. The α-amino groups of amino acid can be protected by various protecting groups, such as phthalyl, formyl, benzyloxycarbonyl, tert-butyloxy (Boc), diphenylisopropyloxycarbonyl (Bpoc), 9-fluorenylmethyloxycarbonyl (Fmoc). In certain embodiments, with amino acids with side chain functional groups, the functional groups can be protected before activation or direct coupling with (−)-huperzine A. The activation of protected amino acids can be performed by conversion of carboxyl groups into acid chlorides, azides, symmetrical anhydrides, mixed anhydrides or active esters by standard methods known to those of skill in the art.

The compounds can be prepared by coupling of activated protected amino acids with (−)-huperzine A or by condensation of protected amino acids directly with (−)-huperzine A or the treatment of dicyclohexylcarbodiimide (DCC) or Bop reagent [Castro, B.;Evin, G.; Selve, R. Tetrahedron lett. 1975, 1219] followed by removal of protecting groups. The methods of deprotection are dependant on the nature of protecting groups, such as hydrazinolysis by treatment of hydrazine, acidolysis by hydrobromic acid in acetic acid, hydrobromic acid in trifluoroacetic acid, or catalyzed hydrolysis. In certain embodiments, more than one amino acid can be linked together to form a peptide chain prior to coupling with (−)-huperzine-A.

Examples of these compounds include: 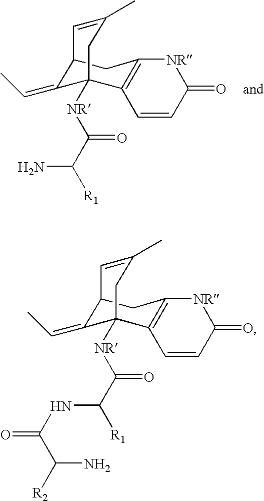
wherein the variables are as described above.

In certain other embodiments, compounds of the present invention represented by formula (II), which are more specifically represented by, for example, 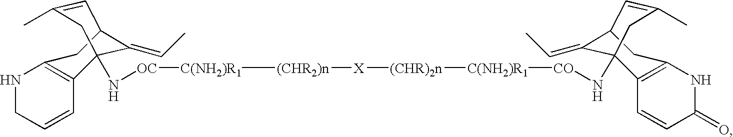
can be prepared by using a dimmer of amino acids, e.g., X[(CHR₂)ₙ—C(NH₂)R₁COOH]₂. Another method of the preparation of these compounds includes first synthesizing compounds such as: 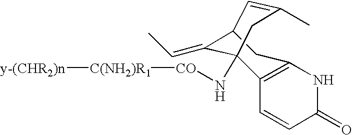
and then linking together two of these compounds.

In another embodiment of the present invention, compounds of formula (II), when R is: 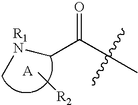
can be synthesized by the methods used in the synthesis described immediately above. The cycloamino groups of amino acids can be protected by formyl, benzyloxycarbonyl, tert-butyloxy (Boc), diphenylisopropyloxycarbonyl (Bpoc), 9-fluorenylmethyloxycarbonyl (Fmoc). The activation of protected amino acids can be performed by conversion of carboxyl groups into acid chlorides, azides, symmetrical anhydrides, mixed anhydrides or active esters. In certain embodiments, the compounds can be prepared by coupling of activated protected amino acids with (−)-huperzine A and followed by deprotection as described immediately above. Examples of these compounds include: 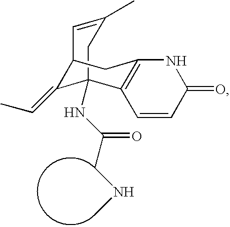
wherein the variables are as described above.

In yet another embodiment of the present invention, compounds of formula (II), when R is —CR₃R₄OC(O)R₅ can be synthesized by condensation of (−)-huperzine A with chloromethyl esters. The later can be prepared by heating the mixture of equivalent of paraformaldehyde, acyl chloride in the presence of catalytic amount of zinc chloride. If aldehydes or ketones are used instead of paraformaldehyde in the reaction of acyl chloride, monosubstituted or disubstituted chloromethyl esters can be obtained. Condensation of (−)-huperzine A with these chloromethyl esters or substituted chloromethyl esters affords N-carboxymethyl-(−)-huperzine A or N-carboxysubstitutedmethyl (−)-huperzine A. Examples of these types of compounds include: 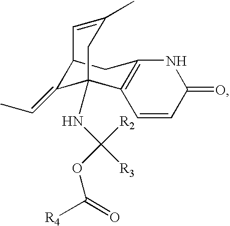
wherein the variables are as described above.

In another embodiment of the present invention, compounds of formula (II), when R is —COOR₅ can be synthesized by the reaction of chlorocarbonates with (−)-huperzine A.

### EXEMPLIFICATION

The invention is further described in the following examples that are in no way intended to limit the scope of the invention.

### Example 1

### Synthesis of L-leucyl-(−)-huperzine A hydrochloride

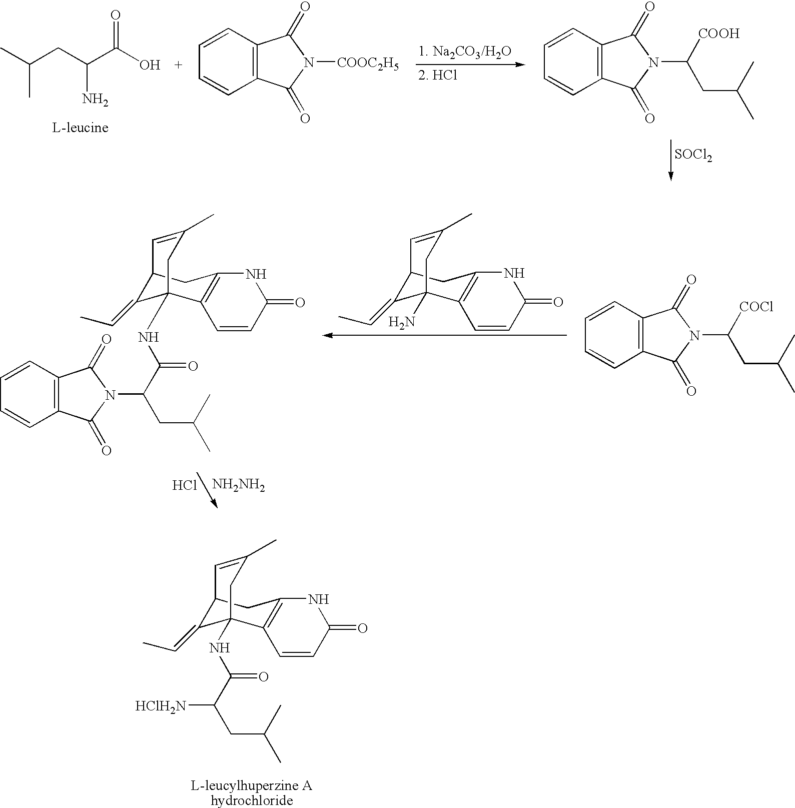 i. Phthalyl-L-leucine:

To a vigorously stirred solution of 1.32 g (10 mmol) of L-leucine, 1.60 g (10 mmol) of sodium carbonate in 15 ml of distilled water was added 2.3 g of powdered N-ethyloxycarbonyl-phthalimide. Almost all of the reagents dissolved in about 15 minutes. The solution was filtered and acidified to pH 2-3 with 6 N hydrochloric acid. The precipitated phthalyl-L-leucine was collected by filtration washed with water and dried in air. It was purified by recrystallization from toluene-hexane to give 2.2 g, m.p. 100°.

ii. Phthalyl-L-leucyl chloride:

A solution of 1.4 g (5 mmole) of phthalyl-L-leucine and 10 ml of freshly distilled thionyl chloride was stirred and gently refluxed under argon atmosphere for 1 hr. The excess thionyl chloride was evaporated under reduced pressure to give crude phthaloyl-leusinyl chloride. The product was coupled with (−)-huperzine A.

iii. Phthelyl-L-leucyl-(−)-huperzine A:

A solution of (−)-huperzine A (24.2 mg, 0.1 mM) and dry triethyl amine (20.2 mg, 0.2 m mole) in 0.5 ml of dry methylene chloride in argon atmosphere was cooled to 0° C. To the solution was added 42 mg of the above phthalyl-leucyl chloride in methylene chloride (0.3 ml) under stirring in argon atmosphere at 0° C. After addition, the reaction mixture was stirred at 0° C. for 30 minutes, and at room temperature for 2 hrs. Subsequently, it was diluted with methylene chloride (5 ml). The whole mixture was washed with water (0.2 ml). After evaporation of solvent under reduced pressure, crude phthalyl-leusinyl (−)-huperzine A was obtained, which was purified by chromatography on silica gel, elution of petane: methylene chloride (4:1) to give phthaloyl-L-leusinyl (−)-huperzine A (34.1 mg, 69.8%).

iv. L-leucyl-(−)-huperzine A hydrochloride:

Phthalyl-leucyl (−)-huperzine A (30 mg, 0.061 mM) and hydrazine (2.5 mg, 0.078 mM) were dissolved in 0.5 ml of absolute ethanol. The solution was refluxed under stirring for 1 hour in argon atmosphere. After removal of ethanol under reduced pressure the residue was stirred with 0.5 ml of 2N hydrochloric acid at room temperature for 30 minutes. The reaction mixture was filtered and separated. The solid was washed with water. The filtrate and washing solution were combined and evaporated to dryness in vacuum below 10° C. to give Leusinyl (−)-huperzine A hydrochloride, white crystals. It was recrystallized from ethanol to afford pure leusinyl (−)-huperzine A hydrochloride.

### Example 2

### Synthesis of L-phenylalanyl-(−)huperzine A hydrobromide

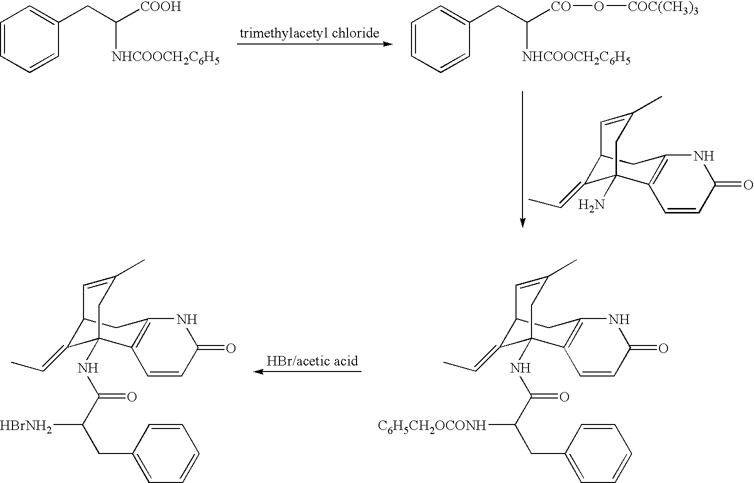 i. Benzyloxycarbonyl-L-phenylalanyl mixed anhydride:

A mixture of 315.2 mg (1 mmole) of benzyloxycarbonyl-L-phenylalanine and 101 mg (1 mM) of triethyl amine in 1 ml of dry toluene was cooled to −5°, and treated with 121 mg (1 mmole) of trimethylacetyl chloride. It was stirred at −5° for 3 hours and at room temperature for 1 hour. The precipitated triethylammonium chloride was removed by filtration and the filtrate was evaporated to dryness in vacuum to give 362 mg of the crude bezyloxycarbonyl-L-phenylalanyl mixed anhydride

ii. Benzyloxycarbonyl-L-phenylalanyl (−)-huperzine A:

A mixture of 42.1 mg (0.11 mmole) of the above mixed anhydride and 24.2 mg (0.1 mmole) of huperzine A in 5 ml of dry toluene in argon atmosphere was stirred and heated at 50° C. for 3 hours and allowed to stand at room temperature overnight. The reaction mixture was diluted with 10 ml of methylene chloride, washed with 0.5 N sodium bicarbonate, 0.5 N hydrochloric acid and water. The solution was dried over magnesium sulfate. Evaporation of solvent under reduced pressure gave the residue, which was chromatographed on silica gel to afford 37 mg of pure benzyloxycarbonyl-L-phenylalanyl (−)-huperzine A.

iii. L-Phenylalanyl (−)-huperzine A hydrobromide:

A suspension of 31 mg (0.06 mmole) of benzyloxycarbonyl-L-phenylalanyl (−)-huperzine A in 0.1 ml of an about 33% solution of hydrobromide in glacial acetic acid was stirred at room temperature for three hours. The solution was evaporated to dryness in vacuum at room temperature to give crude L-Phenylalanyl (−)-huperzine A hydrobromide. Recrystallization from ethanol-ether gave pure L-Phenylalanyl (−)-huperzine A hydrobromide.

### Example 3

### Synthesis of L-threonyl-(−)-huperzine A

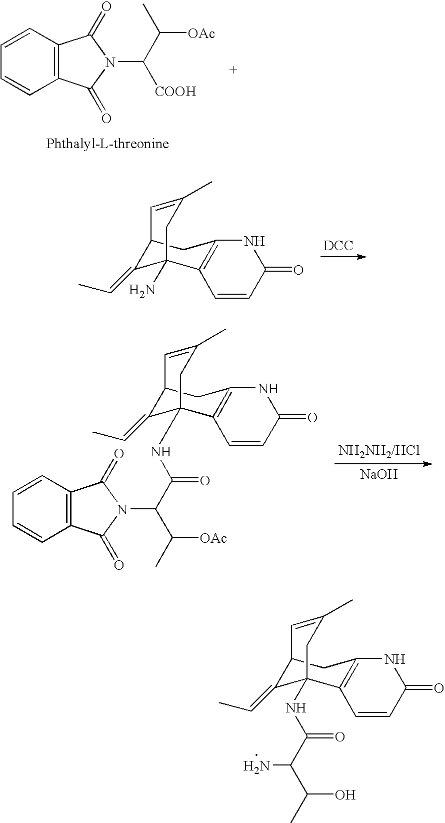 i. O-acetyl-Phthalyl-L-threonyl-(−)-huperzine A:

A mixture of 27.4 mg, (0.11 mmole) of O-acetyl- phthalyl-L-threonine and 24.2 mg, (0.1 mmole) (−)-huperzine A in 0.5 ml of dry methylene chloride in argon atmosphere is cooled to 0° C., to which is added 22.7 mg, (0.11 mmole) of DCC (dicyclohexylcarbodiimide) in 0.3 ml of methylene chloride under stirring in argone atmosphere. After addition, the reaction mixture is stirred at 0° C. for 20 minutes and at room temperature for 3 hrs. Subsequently, it is filtered to remove the separated dicyclohexyl urea. Evaporation of methylene chloride under reduced pressure gives crude product, which is purified by chromatography on silica gel. Elution of petane:methylene chloride (2:1) gives 30 mg of pure O-acetyl-phthalyl-L-threonyl (−)-huperzine A.

ii. L-Threonyl-(−)-huperzine A:

A solution of 30 mg (0.063 mM) of O-acetyl-phthalyl-L-threonyl (−)-huperzine A and hydrazine (2.5 mg, 0.078 mM) in absolute ethanol (0.5 ml) is stirred and refluxed for 1 hr. After removal of ethanol under reduced pressure, the residue is stirred with 2 N hydrochloric acid at room temperature for 30 minutes. The mixture is filtered and separated. The solid is washed with water. The filtrate and washing solution are combined and evaporated to dryness in vacuum. The residue is treated with sodium hydroxide, and then extracted with methylene chloride. Removal of solvent in the residue in reduced pressure gives L-theronyl-(−)-huperzine A,

### Example 4

### Synthesis of L-prolyl-(−)-huperzine A

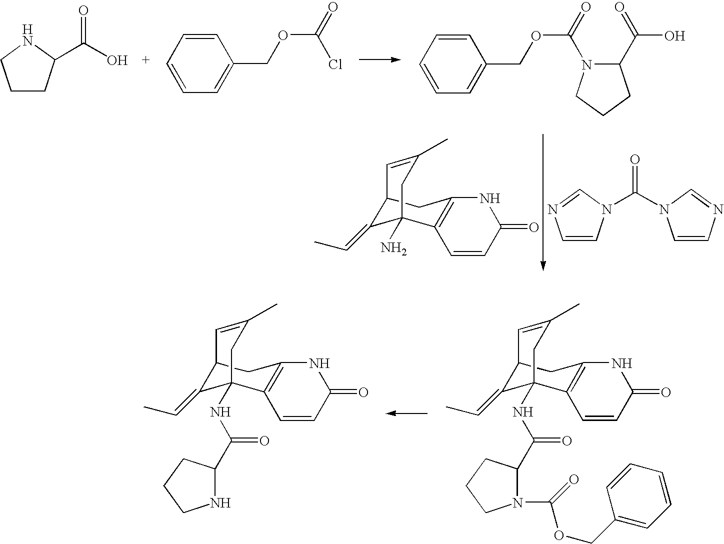 i. Benzyloxycarbonyl-L-proline:

A solution of 11.5 g (100 mmole) of L-proline in 50 ml of 2 N sodium hydroxide, cooled to about 0° C., is added in about ten portions with or without stirring at about 0° C. to the solution of 18.7 g (110 mmole) of benzyl chlorocarbonate and 55 ml of 2 N sodium hydroxide. The reaction mixture should remain distinctly alkaline, if necessary more 2N sodium hydroxide can be added. The temperature of the reaction mixture is kept between 5-10° C. by the rate of addition of the reactants. After addition, the reaction mixture is stirred at room temperature for 30 minutes, and extracted four times with ether. The ether extracts are discarded. The ether dissolved in the aqueous layer is removed by bubbling a stream of nitrogen through the solution. The solution is acidified with 6N hydrochloric acid. An oily substance is separated and slowly solidified to crystal. The crystal is filtered, washed with water and dried in air to give benzyloxycarbonyl-L-proline. It is sufficiently pure for the following reaction.

ii. Benzyloxycarbonyl-L-prolyl-(−)-huperzine A:

To a solution of 27.4 mg (1.1 mmole) of benzyloxycarbonyl-L-proline in 1 ml of dry THF, 16.2 mg of 1,1′-carbonyldiimidazole is added. The solution is stirred at room temperature for 30 minutes, followed by addition of 26.2 mg of (−)-huperzine A. The reaction mixture is allowed to stand at room temperature over night. The solvent is evaporated in vacuum to give the residue. Chromatography of the residue on silica gel gives the pure benzyloxycarbonyl-L-prolyl-huperzine A.

iii. L-prolyl-(−)-huperzine A:

Deprotection of benzyloxycarbonyl-L-prolyl-(−)-huperzine A by hydrobromic acid in glacial acetic acid gives L-prolyl-(−)-huperzine A.

### Example 5

### Synthesis of N-acetoxymethyl-(−)-huperzine

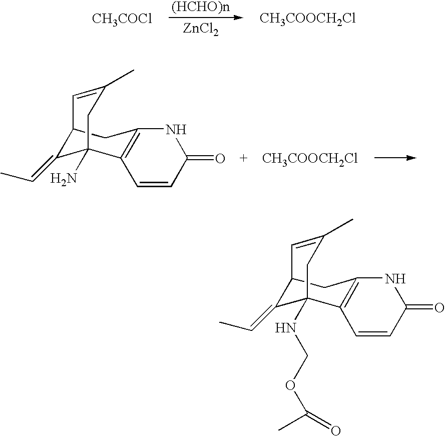 i. Chloromethylacetate:

A mixture of 7.85 g (100 mmole) of acetyl chloride, 3 g (100 mmole) of paraformaldehyde and 10 mg of anhydrous zinc chloride is heated to 60° C. for about 30 minutes and paraformaldehyde is dissolved. The mixture is distilled under reduced pressure (25 mm Hg) and the distillate (b.p. 40-42°/25 mm) is collected by cooling with dry ice. The product, 8.3 g of chloromethyl acetate, is obtained.

ii. N-Acetoxymethyl-(−)-huperzine A:

Chloromethylacetatate is condensed with (−)-huperzine A in the presence of base in methylene chloride. The product is washed with 0.5N hydrochloric acid, water and 0.5N sodium bicarbonate. Evaporation of solvent gives crude product. Chromatography on silica gel affords pure N-Acetoxymethyl-(−)-huperzine A.

### Example 6

### Synthesis of N-benzoyloxymethyl-huperzine

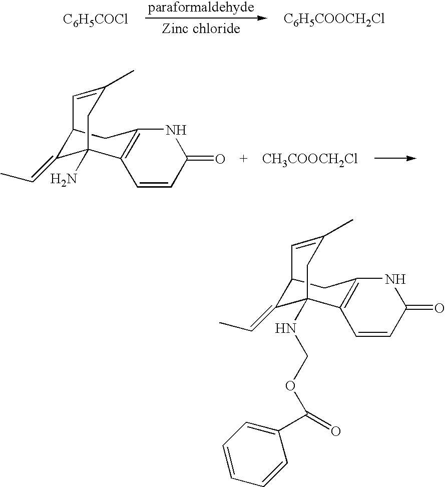 i. Chloromethylbenzoate:

A mixture of 14 g (100 mmole) of benzoyl chloride, 3 g (100 mmole) of paraformaldehyde and 10 mg of anhydrous zinc chloride is heated to 60° C. for about 40 minutes and paraformaldehyde is dissolved. The mixture is distilled in a vacuum. The product, 9.6 g of chloromethyl benzoate (b.p.115-118°/8 mm), is obtained.

ii. N-Benzoyloxymethyl-(−)-huperzine A:

Chloromethylbenzoate is condensed with (−)-huperzine A to give N-benzoyloxymethyl-(−)-huperzine A by the methods described for the preparation of N-acetoxymethyl-(−)-huperzine A

### Example 7

### Synthesis of N-benzoyloxybenzyl-(−)-huperzine

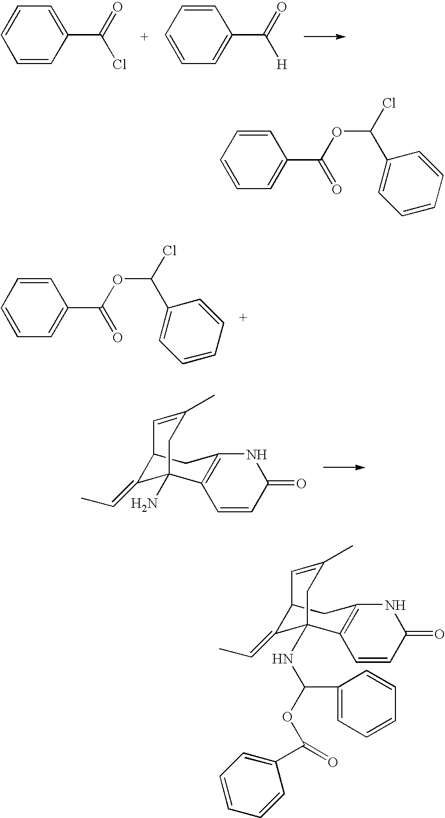 i. Chlorobenzyl benzoate:

A mixture of 14 g (100 mmole) of benzoyl chloride, 10.6 g (100 mmole) of paraformaldehyde and 10 mg of anhydrous zinc chloride is heated to 60° C. for about 40 minutes and paraformaldehyde is dissolved. The mixture is distilled in a vacuum. The product, 12.8 g of chlorobenzybenzoate (b.p.105-108°/0.1 mm), is obtained.

ii. N-Benzoyloxybenzyl-(−)-huperzine A

Chlorobenzyl benzoate is condensed with (−)-huperzine A to give N-benzoyloxymethyl-(−)-huperzine A by the methods described for the preparation of N-acetoxymethyl-(−)-huperzine A to give N-benzoyloxybenzyl-(−)-huperzine A

### Example 8

### Synthesis of N-[(methoxycarbony)oxyl]methyl-huperzine

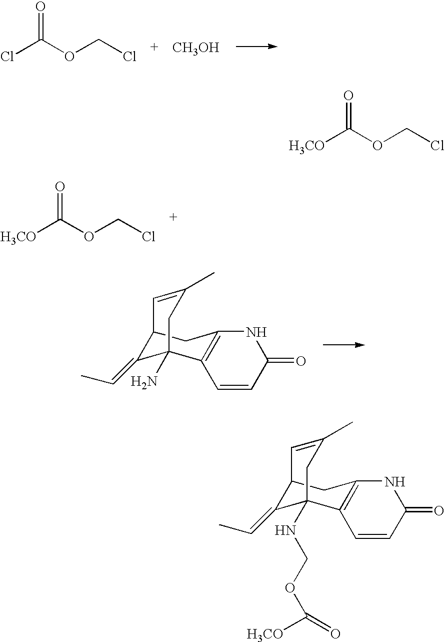 i. Chloromethylmethylcarbonate

A solution of 12.9 g (0.1 mM) of chloromethyl chloroformate in 100 ml of methylene chloride is cooled to −10° C., to which is added a mixture of 3.2 g of methanol and 10.1 g of triethylamine in 40 ml of methylene chloride over 1 hr under stirring. The reaction mixture is stirred at −10° C. for 30 min. and at 0° C. for 1 hr. The precipitate is removed by filtration. The resulting solution is washed with saturated sodium bicarbonate and water, and dried with magnesium sulfate. Evaporation of solvent gives an oily substance, which is purified by distillation and affords chloromethyl methyl carbonate.

ii. N-[(methoxycarbony)oxyl]methyl-huperzine:

This compound is synthesized by the condensation of chloromethylmethylcarbonate with (−)-huperzine A by the methods described for the preparation of N-acetoxymethyl-(−)-Huperzine A.

### Example 9

### Synthesis of Huperzine A Carbonates

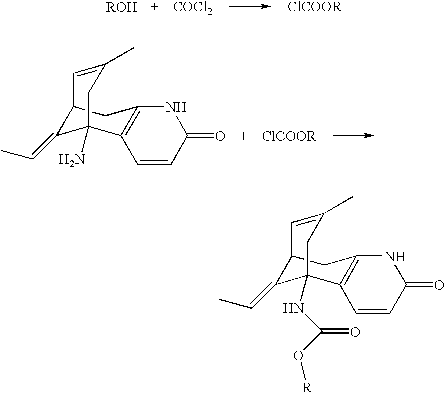 i. Chlorocarabonates:

Some simple chlorocarbonates (such as R=CH₃, C₂H₅, C₃H₇, (CH₃)₃C, C₆H₅, C₆H₅CH₂, etc) are commercially available. The special chlorocarbonates are prepared by reaction of suitable alcohol with phosgene. A solution of 10.9 g (110 mmole) of phosgene in 110 ml of methylene chloride is stirred and cooled to 0-5° C. Subsequently, 100 mmole of suitable alcohol is added to the solution in small portions. After addition the stirring is continued for about 4 hours at 0-5° C. The excess phosgene is removed under reduced pressure to give the chlorocarbonate, which can be purified by distillation, recrcystallization, or used directly without purification.

ii. Huperzine A carbonates:

A suspension of 26.2 mg (0.1 mmole) of (−)-huperzine A in 0.5 ml of 10% solution of sodium carbonate is stirred and cooled at 0-5° C., To the mixture, a solution of 0.12 mmole of chlorocarbonate in 0.3 ml of dioxane is added in small portions and the stirring is continued for 4 hours at 0-5° C. The reaction mixture is diluted with water and then extracted with methylene chloride. The solvent is removed under reduced pressure and the residue is subjected to chromatography on silica gel to give (−)-huperzine A carbonates.

While this invention has been particularly shown and described with references to preferred embodiments thereof, it will be understood by those skilled in the art that various changes in form and details may be made therein without departing from the scope of the invention encompassed by the appended claims.

What is claimed is:

1. A compound represented by the following structural formula: 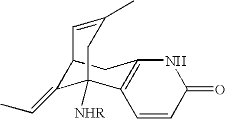
or a pharmaceutically acceptable salt thereof, wherein:
R is:
i) —C(O)C(NR₃R₄)R₁R₂ or —C(O)C[NR₃C(O)C(NR₃R₆)R₁R₂]R₁R₂; 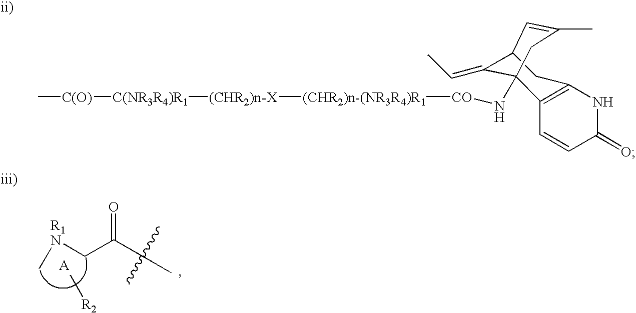
A is a 5, 6 or 7 membered nitrogen containing heterocyclic group;
iv) —CR₃R₄OC(O)R₅;
v) —COOR₅; or 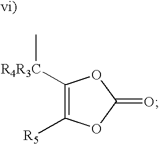
each R₁ is independently —H, C1-20-alkyl, C1-20-aralkyl, aryl, C2-20-alkenyl, C2-20-aralkenyl, C2-20-alknyl, C2-20-aralknyl, carbocyclylalkyl, heterocycloalkyl, C3-20-cycloalkyl, C1-20-hydroxyaralkyl, C1-20-hydroxyalkyl, C1-20-nitroaralkyl, C1-20-nitroalkyl, C1-20-alkoxyalkyl, C1-20-thioalkyl, C1-20-alkylthioalkyl, C1-20-aminoalkyl, C1-20-carboxyalkyl, C1-20-alkoxycarboalkyl, C1-20-aminocarboalkyl, C1-20-guanidinoalkyl, heterocyclyl, C1-20-heterocycloalkyl, C1-20-cycloaminoalkyl;
each R₂, R₃ and R₄ are independently —H, C1-4-alkyl, C1-4-aralkyl, aryl, C2-4-alkenyl, C2-4-alknyl, or —NR₃R₄ forms a 4-6 membered nitrogen containing heterocyclic ring, or —CR₃—R₄ forms a 4-6 membered carbocyclic ring;
each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino;
each R₆ is independently R₄ or —C(O)C(NR₃R₄)R₁R₂;
each n is independently 1, 2, 3, 4, 5, 6, 7 or 8; and
X is —O—, —N(R₂)—, —S—, —(CR₂R₃)ₙ, —O—(CR₂R₃)ₙ—O—, —S—(CR₂R₃)ₙ—S—, —N(R₂)—(CR₂R₃)ₙ—N(R₂)—, —O—C(O)—(CR₂R₃)ₙ—C(O)—O— or —C(O)—O—(CR₂R₃)ₙ—O—C(O)—.

2. The compound of claim 1 wherein R is:
—C(O)C(NR₃R₄)R₁R₂ or —C(O)C[NR₃C(O)C(NR₃R₆)R₁R₂]R₁R₂.

3. The compound of claim 2, wherein R is a natural α-amino-acyl group.

4. The compound of claim 2, wherein R is natural dipeptidyl or tripeptidyl group.

5. The compound of claim 1 wherein R is:

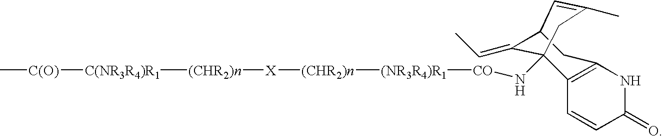

6. The compound of claim 1 wherein R is:

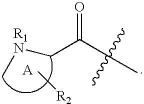

7. The compound of claim 6 wherein R₁ and R₂ are H.

8. The compound of claim 7, wherein A is L-prolyl.

9. The compound of claim 1 wherein R is:
—CR₃R₄OC(O)R₅.

10. The compound of claim 1 wherein R is:
—COOR₅; and
each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino.

11. The compound of claim 1 wherein R is:

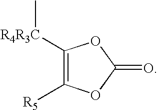

12. A composition comprising a pharmaceutically acceptable carrier or diluent and a compound represented by the following structural formula: 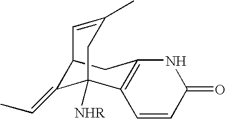
wherein:
R is:
i) —C(O)C(NR₃R₄)R₁R₂ or —C(O)C[NR₃C(O)C(NR₃R₆)R₁R₂]R₁R₂; 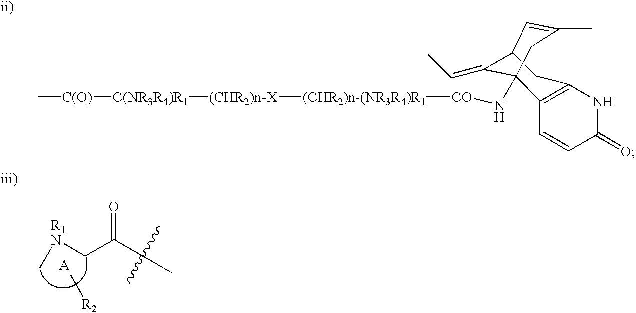
A is a 5, 6 or 7 membered nitrogen containing heterocyclic group;
iv) —CR₃R₄OC(O)R₅;
v) —COOR₅; or 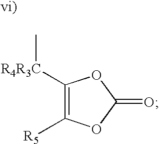
each R₁ is independently —H, C1-20-alkyl, C1-20-aralkyl, aryl, C2-20-alkenyl, C2-20-aralkenyl, C2-20-alknyl, C2-20-aralknyl, carbocyclylalkyl, heterocycloalkyl, C3-20-cycloalkyl, C1-20-hydroxyaralkyl, C1-20-hydroxyalkyl, C1-20-nitroaralkyl, C1-20-nitroalkyl, C1-20-alkoxyalkyl, C1-20-thioalkyl, C1-20-alkylthioalkyl, C1-20-aminoalkyl, C1-20-carboxyalkyl, C1-20-alkoxycarboalkyl, C1-20-aminocarboalkyl, C1-20-guanidinoalkyl, heterocyclyl, C1-20-heterocycloalkyl, C1-20-cycloaminoalkyl;
each R₂, R₃ and R₄ are independently —H, C1-4-alkyl, C1-4-aralkyl, aryl, C2-4-alkenyl, C2-4-alknyl, or —NR₃R₄ forms a 4-6 membered nitrogen containing heterocyclic ring, or —CR₃—R₄ forms a 4-6 membered carbocyclic ring;
each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino;
each R₆ is independently R₄ or —C(O)C(NR₃R₄)R₁R₂;
each n is independently 1, 2, 3, 4, 5, 6, 7 or 8; and
X is —O—, —N(R₂)—, —S—, —(CR₂R₃)ₙ, —O—(CR₂R₃)ₙ—O—, —S—(CR₂R₃)ₙ—S—, —N(R₂)—(CR₂R₃)ₙ—N(R₂)—, —O—C(O)—(CR₂R₃)ₙ—C(O)—O— or —C(O)—O—(CR₂R₃)ₙ—O—C(O)—;
or a pharmaceutically acceptable salt thereof.

13. The composition of claim 12, wherein R is:
—C(O)C(NR₃R₄)R₁R₂ or —C(O)C[NR₃C(O)C(NR₃R₆)R₁R₂]R₁R₂.

14. The composition of claim 13 wherein R is a natural α-amino-acyl group.

15. The composition of claim 13 wherein R is a natural dipeptidyl or tripeptidyl group.

16. The composition of claim 12 wherein R is:

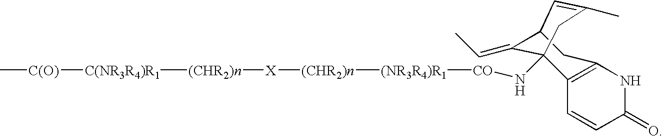

17. The composition of claim 12 wherein R is:

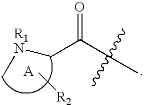

18. The composition of claim 17 wherein R₁ and R₂ are H.

19. The composition of claim 18 wherein A is L-prolyl.

20. The composition of claim 12 wherein R is:
—CR₃R₄OC(O)R₅.

21. The composition of claim 12 wherein R is:
—COOR₅; and
R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino.

22. The composition of claim 12 wherein R is:

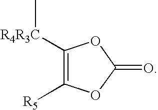

23. The composition of claim 12 wherein the pharmaceutically acceptable carrier is a diluent, an excipient, or a solid carrier, wherein the solid carrier is selected from the group consisting of a gum, a starch, a sugar, a cellulosic material, an acrylate, calcium carbonate, magnesium oxide, talc and mixtures thereof.

24. The composition of claim 12 wherein the pharmaceutically acceptable salt is selected from the group consisting of hydrochloride, hydrobromide, sulfate, nitrate, phosphate, acetate, maleate, formate and tartrate.

25. A method of inhibiting cholinesterase activity in a subject in need thereof, comprising administering to the subject an effective amount of a compound represented by the following structural formula: 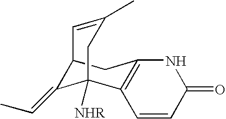
wherein
R is:
i) —C(O)C(NR₃R₄)R₁R₂ or —C(O)C[NR₃C(O)C(NR₃R₆)R₁R₂]R₁R₂; 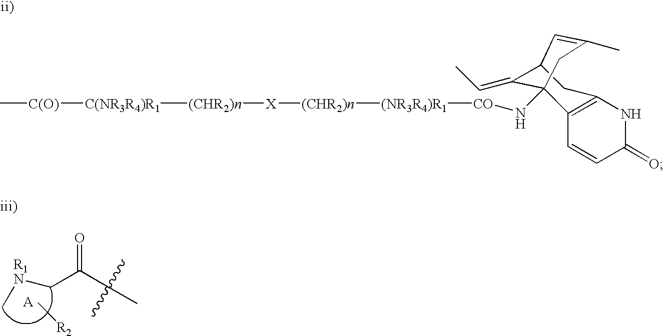
A is a 5, 6 or 7 membered nitrogen containing heterocyclic group;
iv) —CR₃R₄OC(O)R₅;
v) —COOR₅; or
vi) 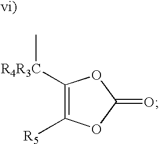
each R₁ is independently H, C1-20-alkyl, C1-20-aralkyl, aryl, C2-20-alkenyl, C2-20-aralkenyl, C2-20-alknyl, C2-20-aralknyl, carbocyclylalkyl, heterocycloalkyl, C3-20-cycloalkyl, C1-20-hydroxyaralkyl, C1-20-hydroxyalkyl, C1-20-nitroaralkyl, C1-20-nitroalkyl, C1-20-alkoxyalkyl, C1-20-thioalkyl, C1-20-alkylthioalkyl, C1-20-aminoalkyl, C1-20-carboxyalkyl, C1-20-alkoxycarboalkyl, C1-20-aminocarboalkyl, C1-20-guanidinoalkyl, heterocyclyl, C1-20-heterocycloalkyl, C1-20-cycloaminoalkyl;
each R₂, R₃ and R₄ are independently —H, C1-4-alkyl, C1-4-aralkyl, aryl, C2-4-alkenyl, C2-4-alknyl, or —NR₃R₄ forms a 4-6 membered nitrogen containing heterocyclic ring, or —CR₃—R₄ forms a 4-6 membered carbocyclic ring;
each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino;
each R₆ is independently R₄ or —C(O)C(NR₃R₄)R₁R₂;
each n is independently 1, 2, 3, 4, 5, 6, 7 or 8; and
X is —O—, —N(R₂)—, —S—, —(CR₂R₃)ₙ, —O—(CR₂R₃)ₙ—O—, —S—(CR₂R₃)ₙ—S—, —N(R₂)—(CR₂R₃)ₙ—N(R₂)—, —O—C(O)—(CR₂R₃)ₙ—C(O)—O— or —C(O)—O—(CR₂R₃)ₙ—O—C(O)—;
and a pharmaceutically acceptable carrier or diluent.

26. A method for treating, preventing or reversing neuronal dysfunction or a neurodegenerative disease in a subject in need thereof, comprising administering to said subject an effective amount of a compound represented by the following structural formula: 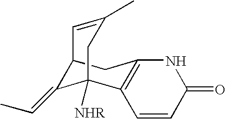
wherein:
R is:
i) —C(O)C(NR₃R₄)R₁R₂ or —C(O)C[NR₃C(O)C(NR₃R₆)R₁R₂]R₁R₂; 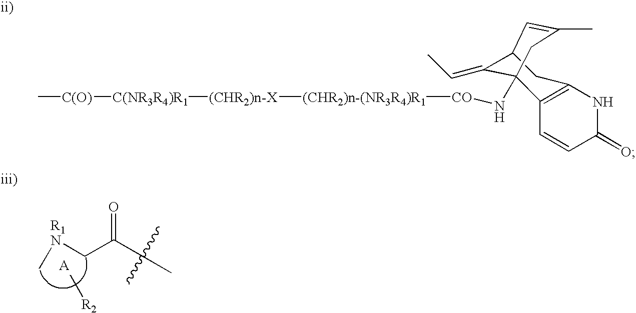
A is a 5, 6 or 7 membered nitrogen containing heterocyclic group;
iv) —CR₃R₄OC(O)R₅;
v) —COOR₅; or 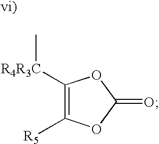
each R₁ is independently —H, C1-20-alkyl, C1-20-aralkyl, aryl, C2-20-alkenyl, C2-20-aralkenyl, C2-20-alknyl, C2-20-aralknyl, carbocyclylalkyl, heterocycloalkyl, C3-20-cycloalkyl, C1-20-hydroxyaralkyl, C1-20-hydroxyalkyl, C1-20-nitroaralkyl, C1-20-nitroalkyl, C1-20-alkoxyalkyl, C1-20-thioalkyl, C1-20-alkylthioalkyl, C1-20-aminoalkyl, C1-20-carboxyalkyl, C1-20-alkoxycarboalkyl, C1-20-aminocarboalkyl, C1-20-guanidinoalkyl, heterocyclyl, C1-20-heterocycloalkyl, C1-20-cycloaminoalkyl;
each R₂, R₃ and R₄ are independently —H, C1-4-alkyl, C1-4-aralkyl, aryl, C2-4-alkenyl, C2-4-alknyl, or —NR₃R₄ forms a 4-6 membered nitrogen containing heterocyclic ring, or —CR₃—R₄ forms a 4-6 membered carbocyclic ring;
each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino;
each R₆ is independently R₄ or —C(O)C(NR₃R₄)R₁R₂;
each n is independently 1, 2, 3, 4, 5, 6, 7 or 8; and
X is —O—, —N(R₂)—, —S—, —(CR₂R₃)ₙ, —O—(CR₂R₃)ₙ—O—, —S—(CR₂R₃)ₙ—S—, N(R₂)—(CR₂R₃)—N(R₂)—, —O—C(O)—(CR₂R₃)ₙ—C(O)—O— or —C(O)—O—(CR₂R₃)ₙ—O—C(O)—;
and a pharmaceutically acceptable carrier or diluent.

27. The method of claim 26, wherein R is:
—C(O)CNR₃R₄)R₁R₂ or —C(O)C[NR₃C(O)C(NR₃R₆)R₁R₂]R₁R₂.

28. The method of claim 27 wherein R is:

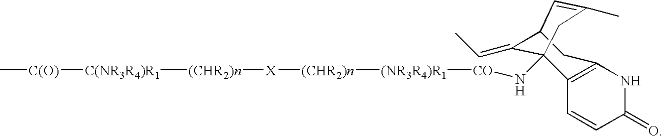

29. The method of claim 26 wherein R is:

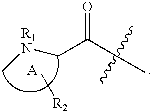

30. The method of claim 26 wherein R is:
—CR₃R₄OC(O)R₅.

31. The method of claim 26 wherein R is:
—COOR₅; and
each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino.

32. The method of claim 26 wherein R is:

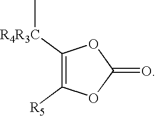

33. The method of claim 26 wherein the neuronal dysfunction is memory impairment.

34. The method of claim 26 wherein the neurodegenerative disease is Alzheimer's Disease.

35. A kit for the treatment, prevention or reversal of a neuronal dysfunction or a neurodegenerative disease, comprising:
I) a compound represented by the following structural formula: 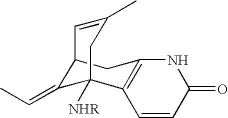
wherein
R is:
i) —C(O)C(NR₃R₄)R₁R₂ or —C(O)C[NR₃C(O)C(NR₃R₆)R₁R₂]R₁R₂; 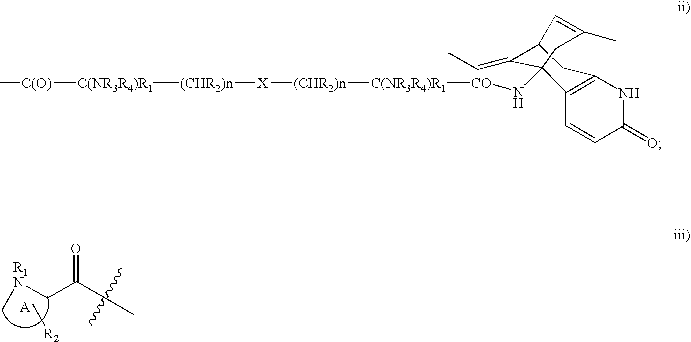
A is a 5, 6 or 7 membered nitrogen containing heterocyclic group;
iv) —CR₃R₄OC(O)R₅;
v) —COOR₅; or
vi) 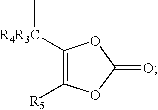
each R₁ is independently H, C1-20-alkyl, C1-20-aralkyl, aryl, C2-20-alkenyl, C2-20-aralkenyl, C2-20-alknyl, C2-20-aralknyl, carbocyclylalkyl, heterocyloalkyl, C3-20-cycloalkyl, C1-20-hydroxyaralkyl, C1-20-hydroxyalkyl, C1-20-nitroaralkyl, C1-20-nitroalkyl, C1-20-alkoxyalkyl, C1-20-thioalkyl, C1-20-alkylthioalkyl, C1-20-aminoalkyl, C1-20-carboxyalkyl, C1-20-alkoxycarboalkyl, C1-20-aminocarboalkyl, C1-20-guanidinoalkyl, heterocyclyl, C1-20-hehertocycloalkyl or C1-20-cycloaminoalkyl;
each R₂, R₃ and R₄ are independently —H, C1-4-alkyl, C1-4-aralkyl, aryl, C2-4-alkenyl, C2-4-alknyl, or —NR₃R₄ forms a 4-6 membered nitrogen containing heterocyclic ring, or —CR₃—R₄ forms a 4-6 membered carbocyclic ring;
each R₅ is independently C1-20-alkyl, C1-20-aralkyl, aryl, C2-20-alkenyl, C2-20-aralkenyl, C2-20-alkynyl, C2-20-aralkynyl, carbocyclyl, carbocyclylalkyl, heterocyloalkyl, C1-20-alkoxyalkyl, C1-20-alkythioalkyl, substituted amino or substituted cycloamino;
each R₆ is independently R₄ or —C(O)C(NR₃R₄)R₁R₂;
each n is independently 1, 2, 3, 4, 5, 6, 7 or 8; and
X is —O—, —N(R₂)—, —S—, —(CR₂R₃)ₙ, —O—(CR₂R₃)ₙ—O—, —S—(CR₂R₃)ₙ—S—, —N(R₂)—(CR₂R₃)—N(R₂)—, —O—C(O)—(CR₂R₃)ₙ—C(O)—O— or —C(O)—O —(CR₂R₃)ₙ—O—C(O)—; and
II) a pharmaceutically acceptable carrier or diluent.

36. The kit of claim 35, further comprising at least one of a radioprotectant, an antimicrobial preservative and a pH-adjusting agent or a filler.

37. The compound of claim 35, wherein R is:
—C(O)C(NR₃R₄)R₁R₂ or —C(O)C[NR₃C(O)C(NR₃R₆)R₁R₂]R₁R₂.

38. The compound of claim 35, wherein R is: [chemical structure]
